(19) United States
(12) Patent Application Publication
(10) Pub. No.: US 2025/0064584 A1
(43) Pub. Date: Feb. 27, 2025

(54) COMMISSURE COVER FOR PROSTHETIC VALVE

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Elena Sherman, Pardes Hana (IL); Anatoly Dvorsky, Haifa (IL); Giolnara Pinhas, Hadera (IL)

(21) Appl. No.: 18/942,285

(22) Filed: Nov. 8, 2024

Related U.S. Application Data
(63) Continuation of application No. PCT/US2023/021785, filed on May 10, 2023.
(60) Provisional application No. 63/341,915, filed on May 13, 2022.

(51) Int. Cl. A61F 2/24

(52) U.S. Cl. CPC A61F 2/2418; A61F 2220/0075; A61F 2250/0036

(57) ABSTRACT

A prosthetic heart valve can include a radially expandable and compressible annular frame, a valvular structure, and a commissure cover. The frame can include a plurality of struts defining the annular frame, and a plurality of commissure support structures each defining a commissure opening positioned along a circumference of the annular frame. The valvular structure can be mounted within the frame, the valvular structure including a plurality of commissures each extending radially through a respective commissure opening to attach the valvular structure to the frame, each commissure having an outflow edge that extends from a radial outer surface of the frame. The commissure cover can be disposed at least partially over the outflow edge of the commissure and can define a ramped surface that extends from the radial outer surface of the frame to a radial outer surface of the commissure.

### CROSS REFERENCE TO RELATED APPLICATIONS

This application is a continuation of International Patent Application No. PCT/US2023/021785, filed on May 10, 2023, which claims the benefit of U.S. Application No. 63/341,915, filed on May 13, 2022. The prior applications are incorporated herein by reference in their entireties.

### FIELD

The present disclosure relates to implantable, mechanically expandable prosthetic devices, such as prosthetic heart valves, and to methods and delivery assemblies for, and including, such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed, for example, for initial loading and/or for repositioning and/or retrieval. However, some components of the prosthetic heart valve can extend past the radial outer surface of the frame when the valve is compressed, causing challenges during loading or re-sheathing.

Despite the recent advancements in percutaneous valve technology, there remains a need for improved transcatheter heart valves and delivery devices for such valves.

### SUMMARY

Described herein are prosthetic heart valves, delivery apparatus, and methods for implanting prosthetic heart valves. The disclosed prosthetic heart valves, delivery apparatus, and methods can, for example, provide cover for components of the prosthetic heart valve that can extend past the radial outer surface of the frame when the valve is compressed. As such, the devices and methods disclosed herein can, among other things, overcome one or more of the deficiencies of typical prosthetic heart valves and their delivery apparatus.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a commissure cover disposed at least partially over the outflow edge of the commissure.

In some examples, a prosthetic heart valve can comprise a commissure cover defining a ramped surface that extends from the radial outer surface of the frame to a radial outer surface of the commissure.

In some examples, the frame of a prosthetic heart valve can comprise a plurality of struts, and one or more struts can comprise eyelets. In some examples, a commissure cover can be coupled to the eyelets via one or more sutures extending through the eyelets.

In some examples, a prosthetic heart valve comprises a commissure cover having a V-shaped cutout at an outflow end portion, the outflow end portion being sutured to one or more struts of the frame along the V-shaped cutout.

In some examples, a prosthetic heart valve comprises a commissure cover comprising a ramp member.

In some examples, a prosthetic heart valve comprises one or more of the components recited in Examples 1-69 below.

In a representative example, a prosthetic heart valve can comprise a radially expandable and compressible annular frame, a valvular structure, and a commissure cover. The frame can have an inflow end, an outflow end, a plurality of struts defining the annular frame, and a plurality of commissure support structures each defining a commissure opening positioned along a circumference of the annular frame. The valvular structure can be mounted within the annular frame and can comprise a plurality of leaflets and a plurality of commissures each extending radially through a respective commissure opening to attach the valvular structure to the frame, each commissure having an outflow edge that extends from a radial outer surface of the frame. The commissure cover can be disposed at least partially over the outflow edge of the commissure, the commissure cover defining a ramped surface that extends from the radial outer surface of the frame to a radial outer surface of the commissure.

In another representative example, a prosthetic heart valve can comprise a radially expandable and compressible annular frame, a valvular structure, and a commissure cover. The frame can have an inflow end, an outflow end, a plurality of struts defining the annular frame, and a plurality of commissure support structures each defining a commissure opening positioned along a circumference of the annular frame, wherein one or more selected struts of the plurality of struts comprise eyelets. The valvular structure can be mounted within the annular frame and can comprise a plurality of leaflets and a plurality of commissures each extending radially through a respective commissure opening to attach the valvular structure to the frame, each commissure having an outflow edge that extends from a radial outer surface of the frame. The commissure cover can be disposed at least partially over the outflow edge of the commissure, the commissure cover having a first end portion and a second end portion and defining a ramped surface that extends from the radial outer surface of the frame to a radial outer surface of the commissure when the commissure cover is coupled to the frame. The prosthetic valve can further comprise an outer skirt disposed on the radial outer surface of the frame. The first end portion of the commissure cover can be coupled to the eyelets by one or more sutures; and the second end portion of the commissure cover can be coupled to the outer skirt by one or more sutures.

In another representative example, a prosthetic heart valve can comprise a radially expandable and compressible annular frame, a valvular structure, and a commissure cover. The frame can have an inflow end, an outflow end, a plurality of struts defining the annular frame, and a plurality of commissure support structures each defining a commissure opening positioned along a circumference of the annular frame. The valvular structure can be mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame. The commissure cover can comprise a plurality of fabric layers arranged to define a ramp member, the commissure cover disposed adjacent the outflow edge of the commissure, wherein the radial outer surface of the commissure cover defines a ramped surface that extends from the radial outer surface of the frame to a radial outer surface of the commissure.

In yet another representative example, a prosthetic heart valve can comprise a radially expandable and compressible annular frame, a valvular structure, an outer skirt, and a commissure cover. The frame can have an inflow end, an outflow end, a plurality of struts defining the annular frame, and a plurality of commissure support structures each defining a commissure opening positioned along a circumference of the annular frame. The valvular structure can be mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame. The commissure cover can have a first end portion and a second end portion, the first end portion being coupled to the outer skirt via one or more sutures, the second end portion being folded to form a plurality of layers arranged to define a ramp member, the second end portion being positioned upstream adjacent the outflow edge of the commissure to define a ramped surface from the radial outer surface of the frame to the radial outer surface of the commissure

In still another representative example, a prosthetic heart valve can comprise a radially expandable and compressible annular frame, a valvular structure, and a commissure cover. The frame can have an inflow end, an outflow end, a plurality of struts defining the annular frame, and a plurality of commissure support structures each defining a commissure opening positioned along a circumference of the annular frame. The valvular structure can be mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame. The commissure cover can comprise a rigid ramp member including one or more openings extending through a thickness of the ramp member, the ramp member disposed adjacent the outflow edge of the commissure and including a ramped surface that extends from the radial outer surface of the frame to the radial outer surface of the commissure.

In another representative example, a prosthetic heart valve can comprise a radially expandable and compressible annular frame, a valvular structure, and one or more guide sutures. The frame can have an inflow end, an outflow end, a plurality of struts defining the annular frame, and a plurality of commissure support structures each defining a commissure opening positioned along a circumference of the annular frame, selected struts of the plurality of struts comprising eyelets extending from an inflow surface of the strut toward an inflow end portion of the frame. The valvular structure can be mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame. The one or more guide sutures can extend through a respective commissure and be coupled to the eyelets of the frame such that the guide sutures define a ramped surface from the eyelets to a radial outer surface of the commissure.

In a representative example, a method of loading a prosthetic heart valve into a shaft can comprise radially compressing a prosthetic heart valve, the prosthetic heart valve comprising an annular frame having an inflow end, an outflow end, a plurality of struts, and a plurality of commissure support structures each defining a commissure opening positioned along a circumference of the annular frame, a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame, and a commissure cover disposed at least partially over the outflow edge of the commissure. The method can further comprise as the frame is radially compressed, allowing the commissure cover to be pulled taut to define a ramped surface that extends from the radial outer surface of the frame to a radial outer surface of the commissure, and advancing a shaft of a delivery apparatus over the radially compressed prosthetic valve such that a distal edge of the shaft advances along the ramped surface and does not engage the outflow edge of the commissure.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2 is a front portion of the frame for the prosthetic valve of FIG. 1A.

FIG. 3 is a side elevation view of a delivery apparatus for a prosthetic device, such as a prosthetic valve, according to one example.

FIG. 4 is a side elevation view of one example of a commissure of a prosthetic valve.

FIG. 5 is a perspective view of one example of a prosthetic heart valve including the commissure of FIG. 4 being loaded into a sheath.

FIG. 6 is a perspective view of an exemplary commissure cover mounted on an exemplary prosthetic heart valve.

FIG. 7 is a perspective view of an exemplary commissure cover mounted on an exemplary prosthetic heart valve.

FIG. 8 is a partial cross-section side view of a portion of a prosthetic heart valve including the commissure cover of FIG. 6.

FIGS. 9-10 are perspective views of another exemplary commissure cover mounted on a prosthetic heart valve.

FIG. 11 is a partial cross-section side view of a portion of yet another example of a commissure cover mounted on a prosthetic heart valve.

FIGS. 12-14 are perspective views of an exemplary commissure cover mounted on a prosthetic heart valve.

FIGS. 15-16 are perspective views of another exemplary commissure cover mounted on a prosthetic heart valve.

FIGS. 17-19 are perspective views of the commissure cover of FIG. 15 including exemplary guide sutures mounted on a prosthetic heart valve.

FIGS. 20-23 are perspective views of exemplary guide sutures mounted on a prosthetic heart valve.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

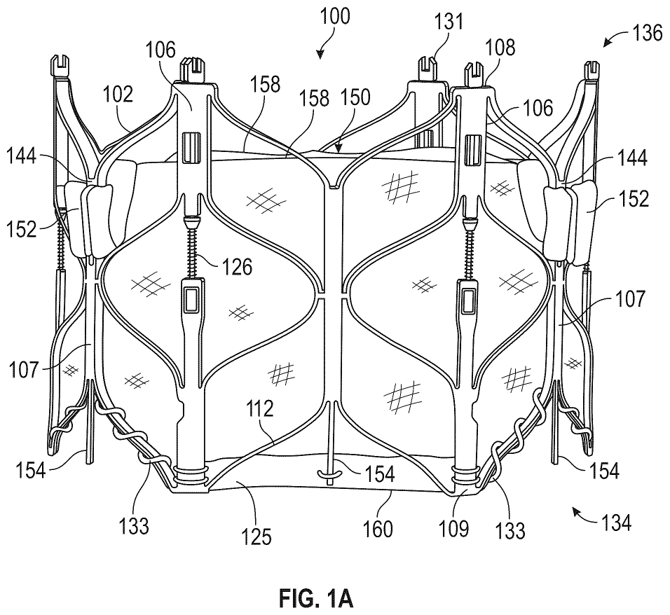

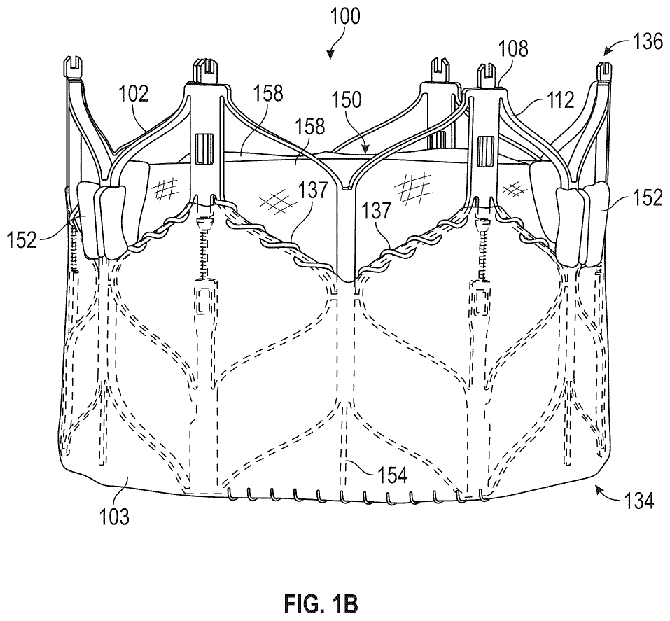

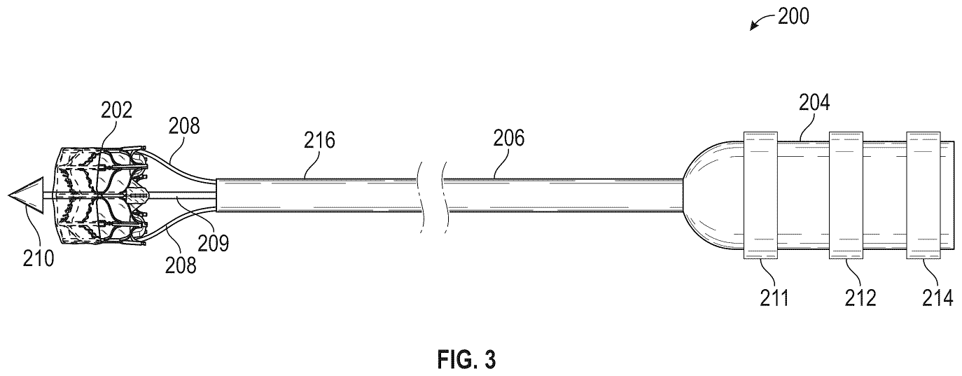

FIGS. 1A-1B illustrate an exemplary prosthetic device (for example, prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

### Examples of the Disclosed Technology

FIGS. 1A-1B show an exemplary prosthetic valve 100, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Pat. No. 10,363,130, which is incorporated by reference herein in its entirety. In another example, the disclosed prosthetic valves can be implanted within a docking device implanted within or at the native mitral valve, such as disclosed in PCT Publication No. WO2020/247907, which is incorporated herein by reference in its entirety. In another example, the disclosed prosthetic valves can be implanted within a docking device implanted within the superior or inferior vena cava for replacing the function of a diseased tricuspid valve, such as disclosed in U.S. Pat. No. 11,291,540, which is incorporated herein by reference in its entirety.

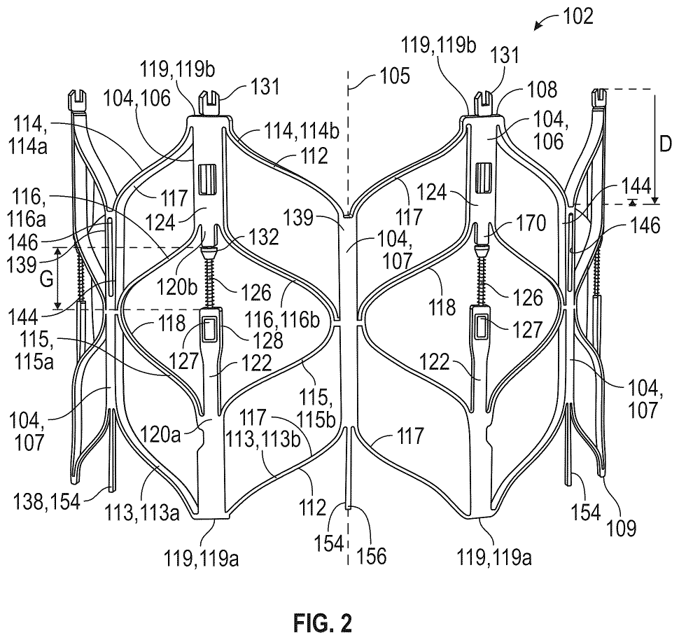

FIGS. 1A-2 illustrates an exemplary example of a prosthetic valve 100 (which also may be referred to herein as “prosthetic heart valve 100”) having a frame 102. FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2, it should be appreciated that the frame 102 forms an annular structure having an opposite side that is substantially identical to the portion shown.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In some examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Pat. No. 11,399,932, which is incorporated herein by reference in its entirety.

Further details regarding the assembly of the leaflet assembly and the assembly of the leaflets and the skirts to the frame can be found, for example, in International Publication Nos. WO2022/261419 and WO2023/003696, which are incorporated herein by reference in their entireties. Further details of the construction and function of the frame 102 can be found in International Publication No. WO2022/072564, which is incorporated herein by reference in its entirety.

The frame 102, which is shown alone and in greater detail in FIG. 2, comprises an inflow end 109, an outflow end 108, and a plurality of axially extending posts 104. The axial direction of the frame 102 is indicated by a longitudinal axis 105, which extends from the inflow end 109 to the outflow end 108. Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in some examples.

As illustrated in FIG. 2, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIG. 2, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIG. 2, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2) (those with actuators 126 can be referred to as actuator posts or actuator struts). Each first post 122 (that is, the lower post shown in FIG. 2) can extend axially from the inflow end 109 of the prosthetic valve 100 toward the second post 124, and the second post 124 (that is, the upper post shown in FIG. 2) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In some examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In some examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2) separating the posts 122, 124, and thereby radially compressing or radially expanding the prosthetic valve 100, respectively. Thus, the gap G (FIG. 2) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIG. 2). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In some examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIG. 2, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in some examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance D (indicated in FIG. 2). As an example, the offset distance D may be in a range from 2 mm to 6 mm. In general, the offset distance D should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in some examples, the frame 102 can comprise more or less than six support posts 107 and/or more or less than three commissure support structures 144.

The inflow end portion 138 of each support post 107 can comprise an extension 154 (show as a cantilevered strut in FIG. 2) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156. Further details of the extensions 154 and their use can be found, for example in International Publication No. WO2022/072564, which is incorporated by reference herein in its entirety.

As mentioned previously, in some examples the prosthetic heart valve 100 can comprise an outer skirt 103 mounted around the outer surface of frame 102 as shown in FIG. 1B, and/or a connecting skirt 125 (FIG. 1A) and/or an inner skirt. Further details of the skirts can be found, for example, in International Publication No. WO2022/072564.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In some examples, the frame 102 can be formed from a self-expandable material (for example, Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. FIG. 3 illustrates an exemplary delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In some examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In some examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 202, the delivery apparatus 200 can be removed from the patient, leaving just the prosthetic valve 202 in the patient.

Further details of the delivery apparatus 200, including structures and methods of coupling the prosthetic valve 202 to the delivery apparatus 200 and releasing the prosthetic valve therefrom, can be found, for example, in International Publication No. WO2022/072564, which is incorporated by reference herein in its entirety.

If repositioning or recapture and removal of the prosthetic valve 202 is desired, the prosthetic valve can be compressed (from an expanded or partially expanded configuration) by rotating the threaded rods of the prosthetic valve 202 in a second, opposing direction (for example, using second knob 212). Once the prosthetic valve 100 has been recompressed it can be repositioned at the implantation site, once repositioned, the prosthetic valve 202 can be expanded as described previously. The prosthetic valve can be re-compressed, repositioned, and re-expanded multiple times, as needed. In some cases, the prosthetic valve 202 can be fully compressed and “recaptured,” that is, retracted back into a sheath and/or removed from the patient's body.

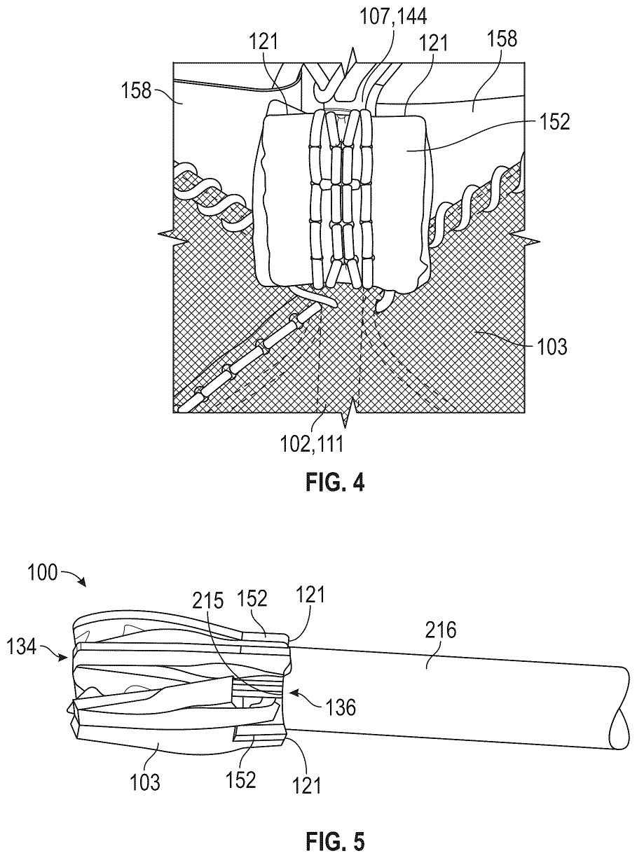

Referring to FIGS. 4-5, in some examples, the portions of the commissures 152 that extend through the commissure opening 146 (FIG. 2) of the frame 102 can form a step-wise configuration relative to a radial outer surface 111 of the frame 102. In other words, the outflow edge 121 (FIG. 4) of each commissure 152 can extend from the radial outer surface 111 of the frame 102 (for example, in a substantially perpendicular direction) forming a lip or shoulder. As shown in FIG. 5, sheathing the prosthetic valve 100 within the capsule or sheath 216 (for example, to allow for initial crimping, repositioning of the prosthetic valve, or removal of the prosthetic valve from the subject's body) can be challenging if the outflow edges 121 of one or more commissures engage or abut a distal edge 215 of the capsule or sheath 216.

Though the examples disclosed herein refer to commissure covers and/or guide sutures for covering some or all of a commissure (such as commissure 152), the commissure covers and/or guide sutures described herein can also be used to cover and form a ramped surface over any feature that extends radially outwardly of the frame, for example, to prevent the sheath 216 from engaging that feature when the prosthetic valve is being loaded into the sheath. For example, commissure covers/guide sutures such as those described herein can be used to cover the attachment of the scallop line to the frame, if such attachment is positioned on the radial outer surface of the frame.

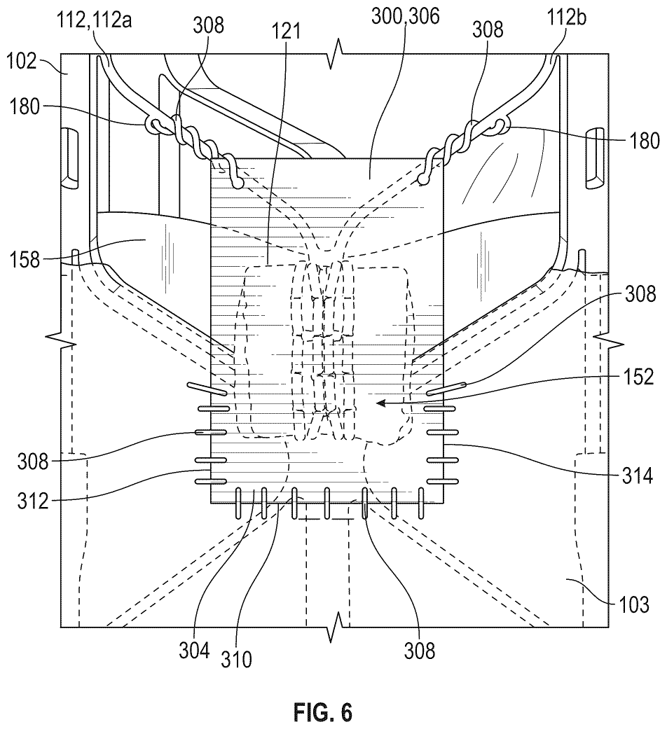

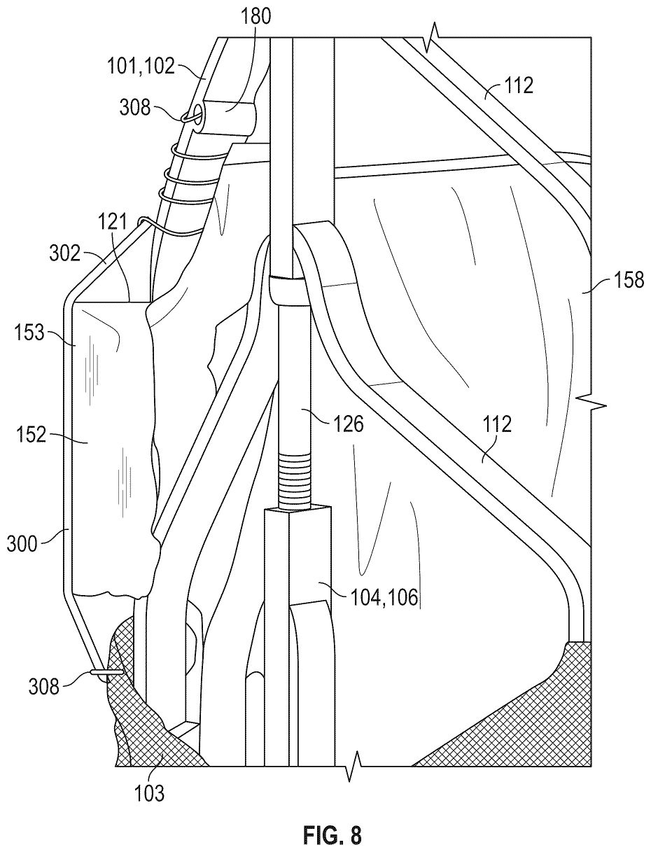

FIGS. 6-8 illustrate an exemplary commissure cover 300 configured to mitigate or prevent engagement of the outflow edge 121 of the commissure 152 with the distal edge of the sheath 216. Referring to FIG. 6, the commissure cover 300 can be a member configured (for example, sized and shaped) such that when the commissure cover 300 is coupled to the prosthetic valve 100 it extends over the outflow edge 121 of the commissure 152, creating a ramped outer surface 302 (FIG. 8) from the radial outer surface 101 of the frame 102 to a radial outer surface 153 of the commissure 152.

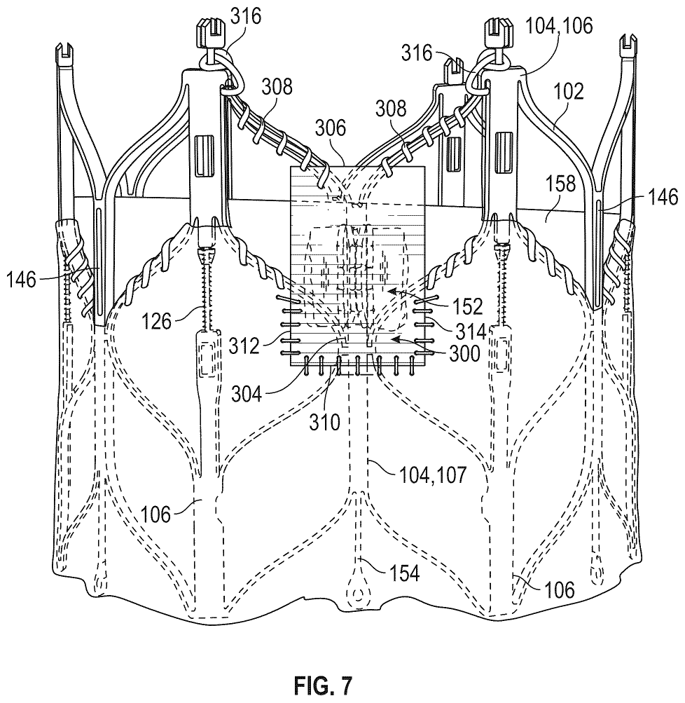

Though FIG. 7 shows only a single commissure cover 300 for purposes of illustration, it should be understood that a commissure cover 300 can be provided for each commissure on the prosthetic valve. For example, three commissure covers 300 can be provided for a prosthetic valve having three commissures (such as prosthetic valve 100 shown in FIGS. 1A-1B). In some examples, a greater or fewer number of commissures can be present and thus a greater or fewer number of commissure covers can be used.

The cover 300 can comprise any of various suitable biocompatible materials, including any of various synthetic materials, including fabrics (for example, polyethylene terephthalate fabric) or natural tissue (for example, pericardial tissue). In some examples, the commissure covers 300 can comprise woven PET fabric.

Referring still to FIG. 6, the cover 300 can have a first end portion configured as an inflow end portion 304, a second end portion configured as an outflow end portion 306. The inflow end portion 304 of the cover can be coupled to a skirt of the frame, for example the outer skirt 103 described previously. In some examples, the inflow end portion 304 can be coupled to an inner skirt. In some examples, the inflow end portion 304 can be coupled to the outer surface of the commissure 152, and/or to the inflow edge 121 of the commissure.

As shown in FIG. 6, the inflow end portion 304 can be coupled to the outer skirt 103 via one or more sutures 308 extending through the commissure cover 300 and the outer skirt 103. In some examples, the sutures 308 can be stitched in a whipstitch pattern or running stitch pattern. In the illustrated example, the inflow end portion 304 of the cover 300 comprises an inflow edge 310, a first side edge 312, and a second side edge 314. In some examples, such as the illustrated example shown in FIG. 6, the inflow edge 310 and at least a portion of each side edge 312, 314 can be coupled to the outer skirt 103. In some examples, only the inflow edge 310 can be coupled to the outer skirt 103, or only the side edges 312, 314, or any combination of the side edges and inflow edge.

The outflow end portion 306 can be coupled directly to the frame 102, for example, using one or more sutures 308 to couple the cover 300 to selected struts 112. In the example illustrated in FIGS. 6-8, the outflow end portion 306 is coupled to a first strut 112a at a first selected location (for example, a first corner of the cover 300) and to a second strut 112b at a second selected location (for example, a second corner of the cover 300). In the illustrated example the first and second struts 112a, 112b are disposed at the outflow end 136 (FIG. 1B) of the frame 102. However, in some examples, the struts 112a, 112b can be disposed at any axial location along the frame 102 (for example, between the outflow end 136 and the inflow end 134). The sutures 308 can extend around the struts 112 in a helical pattern.

In some examples, as shown in FIGS. 6 and 8, each selected strut 112 can comprise an eyelet 180 to which a respective suture can be secured (for example, by knotting). The eyelet 180 can extend from a surface of the strut 112, for example, the surface facing the inflow end, as shown in FIG. 6. In some examples, the eyelet 180 can extend from the surface facing the outflow end, or from a side surface of the strut 112. In some examples, the eyelet 180 can be an aperture or opening extending through a thickness of the strut 112 in the axial or radial direction. In another example, as shown in FIG. 7, the sutures 308 can extend around the struts 112 and be coupled to the outflow end of the adjacent posts 104, for example, by securing sutures around the head portion 131 (FIG. 2) of the threaded rod 126 with a knot 316.

The outflow end portion 306 of the cover 300 can be coupled to the frame 102 such that when the frame 102 is in the radially expanded configuration (for example, as shown in FIGS. 6-7), the sutures 308 coupling the cover 300 to the frame 102 are not pulled tight. When the frame 102 is radially compressed, the eyelets 180 move away from the outflow end 306 of the cover 300, thereby maintaining or increasing the tightness of the sutures 308 such that the cover 300 is pulled taut over the commissure 152, creating or maintaining a ramped surface. Referring to FIG. 8, the radial outer surface of the cover 300 forms the taut, ramped surface 302 such that the sheath 216 can advance along the ramped surface 302 and over the commissure 152 without engaging the outflow edge 121 of the commissure.

In the example illustrated in FIGS. 6-8, the cover 300 has a rectangular shape. In some examples, the cover 300 can have any shape configured to cover at least the outflow edge 121 of the commissure 152, such as square, oval, triangular, circular, etc.

In the illustrated example, the cover 300 is a separate piece of material coupled to the outer skirt 103 and the frame 102. However, in some examples, the cover 300 can be formed integrally with the skirt 103, for example, as a flap or folded portion of the skirt that extends over the commissure 152 and is secured to the frame. In some examples, the commissures 152 can each comprise a flexible connector used to secure adjacent sides of the leaflets to one another. Examples of such flexible connectors can be found, at least, in U.S. Pat. No. 9,393,110, which is incorporated by reference herein in its entirety. In such examples, the commissure cover 300 can be coupled to or formed integrally as part of the flexible connector. For example, the commissure cover 300 can be a flap coupled to the flexible connector at the inflow end portion (for example, via sutures or formed integrally with the flexible connector) that folds over the commissure 152 and is coupled to the frame 102 of the prosthetic valve.

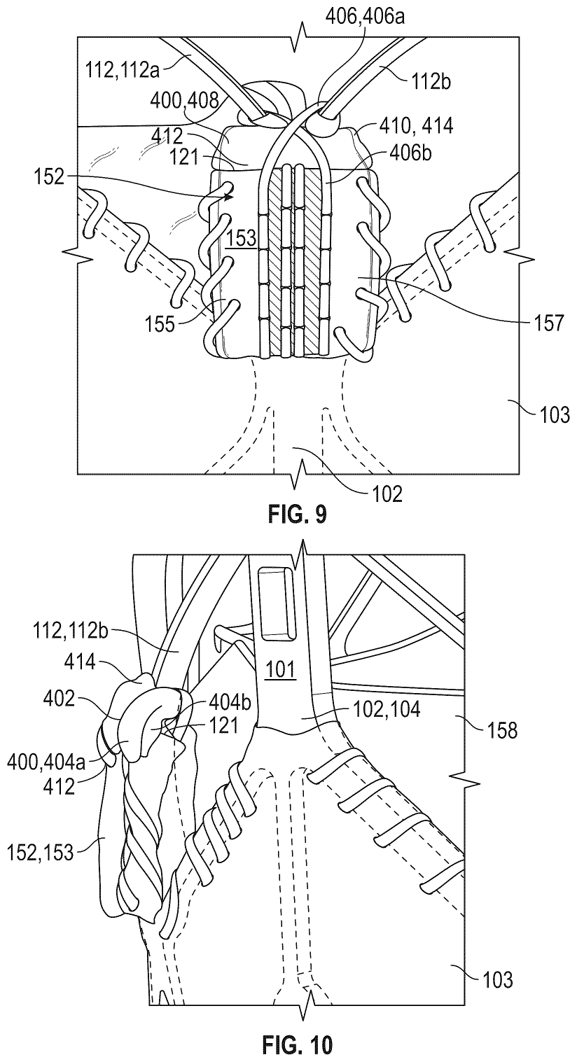

Referring to FIGS. 9-10, in another example, in lieu of or in addition to commissure cover 300, each commissure 152 can comprise a commissure cover 400. As shown in FIG. 9, commissure cover 400 can be coupled to the prosthetic valve 100 such that it covers at least a portion of the commissure 152 (for example, the outflow edge 121) and creates a ramped outer surface 402 (FIG. 10) from the radial outer surface 101 of the frame 102 to the radial outer surface 153 of the commissure 152.

The commissure cover 400 can comprise a rolled or folded piece of material. For example, as shown in FIG. 10, the commissure cover 400 can be a piece of suitable biocompatible material that has been folded and positioned adjacent and/or partially over the outflow edge 121 of the commissure. The cover 400 can comprise any of various suitable biocompatible materials, including any of various synthetic materials, including fabrics (for example, polyethylene terephthalate fabric) or natural tissue (for example, pericardial tissue). In some examples, the commissure covers 400 can comprise woven PET fabric.

In the illustrated example, the cover 400 is a rectangular piece of material that has been folded such that it has at least two layers 404a, 404b. As can be seen in FIG. 10, the cover 400 can be positioned such that it at extends at least partially over the outflow edge portion 121 of the commissure 152. In some examples, the cover 400 can extend at least partially over the radial outer surface 153 of the commissure 152 as well.

Referring to FIG. 9, the cover 400 can be coupled to the commissure 152 in the following exemplary manner. A first suture 406a can extend through a first side portion 155 of the commissure 152 and/or a first side portion 408 of the commissure cover 400. The first suture 406a can extend around a first selected strut 112a and then be sewn through the second side portion 157 of the commissure 152. A second suture 406b can extend through the second side portion 157 of the commissure 152 and/or a second side portion 410 of the commissure cover 400, around a second selected strut 112b and then through the first side portion 155 of the commissure 152, such that the first and second sutures 406 form an X-shape over the commissure cover 400.

In the illustrated example, the first and second sutures 406 are coupled to the selected struts 112 by wrapping the sutures around each strut. However, in some examples, the sutures 406 can be coupled to an eyelet formed as part of the frame 102, such as eyelet 180 in FIG. 6.

In some examples, an inflow end portion 412 of the commissure cover 400 can be sutured to an outflow end portion of the commissure (for example, outflow edge 121), and an outflow end portion 414 of the cover 400 can be secured to the frame 102 by sutures extending through the cover 400 radially inwardly through the commissure window 146. In some examples, the sutures extending through the commissure window 146 can be coupled to a wedge member (that is, a thick suture) disposed on a radial inner surface of the frame 102. Such a configuration can mitigate or retain the commissure cover 400 against axial movement relative to the commissure and/or from radial movement relative to the frame. When the prosthetic valve 100 is radially compressed, the sheath 216 can advance over the ramped surface created by the cover 400.

In the illustrated example the cover 400 is shown as a separate component. However, in some examples, the cover 400 can be formed integrally with other components of the prosthetic valve, such as described previously with respect to cover 300.

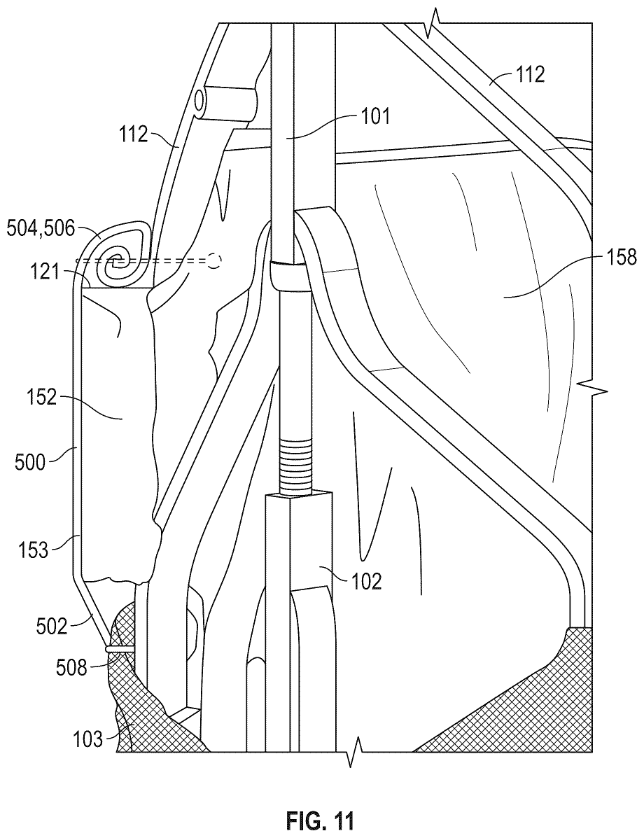

Referring to FIG. 11, in some examples, in lieu of or in addition to commissure covers 300 and/or 400, the prosthetic valve 100 can comprise commissure cover 500. Commissure cover 500 can be an elongated member having a first or inflow end portion 502 coupled to the outer skirt 103 using one or more sutures 508 (for example, as described previously for inflow end portion 304 of commissure cover 300). The second end portion 504 of the elongated member can be rolled or folded to form a wedge-shaped member 506 similar to commissure cover 400. The wedge member 506 can be disposed on the outflow edge 121 of the commissure 152. When the frame 102 is radially compressed, the wedge-shaped member 506 forms a ramp from the radial outer surface 101 of the frame 102 to the radial outer surface 153 of the commissure 152. The sheath 216 can then advance over the ramped surface (for example, the ramp formed by the wedge-shaped member 506) without catching on the outflow edge 121 of the commissure.

As shown in FIG. 11, the second end portion 504 can be coupled to the frame 102, for example, using one or more sutures 508 extending through the wedge-shaped member 506 and through the commissure window 146 (FIG. 2) in the frame 102.

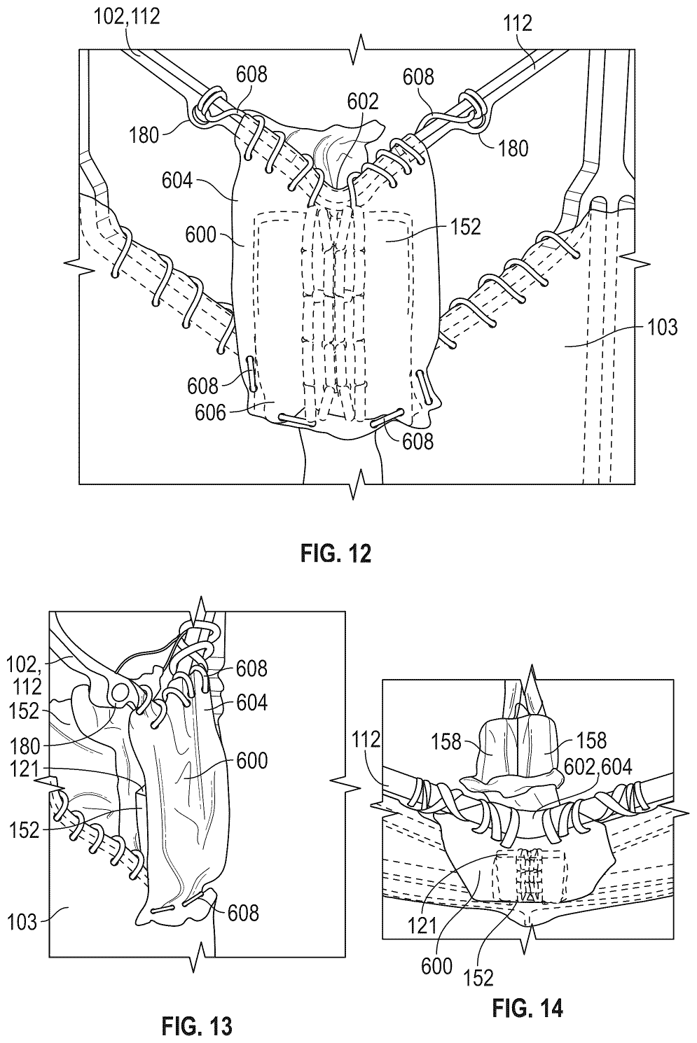

FIGS. 12-14 illustrate another example of a commissure cover 600. Commissure cover 600 is similar to commissure cover 300 described previously, except that the outflow end portion 604 of commissure cover 600 is sutured to the struts 112 of the frame in a V-shaped pattern. As shown in FIG. 12, an inflow end portion 606 of the commissure cover 600 can be coupled to the outer skirt 103 using one or more sutures 608 (for example, as described previously for inflow end portion 304 of commissure cover 300), and the outflow end portion 604 can be coupled to the frame 102. One or more sutures 608 can be sewn through the outflow end portion 604 and wrapped around one or more selected struts 112 to form a V-shaped suture pattern, for example, as shown in FIGS. 12-14. In some examples, the sutures 608 can be coupled to eyelets 180 extending from the struts 112. In some examples, the outflow end portion 604 can include a V-shaped opening or cutout 602 in the outflow end portion 604 and the commissure cover 600 can be sutured to the frame along the V-shaped opening 602.

The outflow end portion 604 of the cover 600 can be coupled to the frame 102 such that when the frame 102 is in the radially expanded configuration (for example, as shown in FIGS. 12-14), the cover 600 is not necessarily pulled taut. When the frame 102 is radially compressed, the eyelets 180 move away from the outflow edge 121 of the commissure 152, thereby maintaining or increasing the tightness of the sutures 608 such that the cover 600 is pulled taut over the outflow edge 121. The radial outer surface of the cover 600 creates or maintains a taut, ramped surface such that the sheath 216 can advance along the ramped surface and over the commissure 152 without engaging the outflow edge 121 of the commissure.

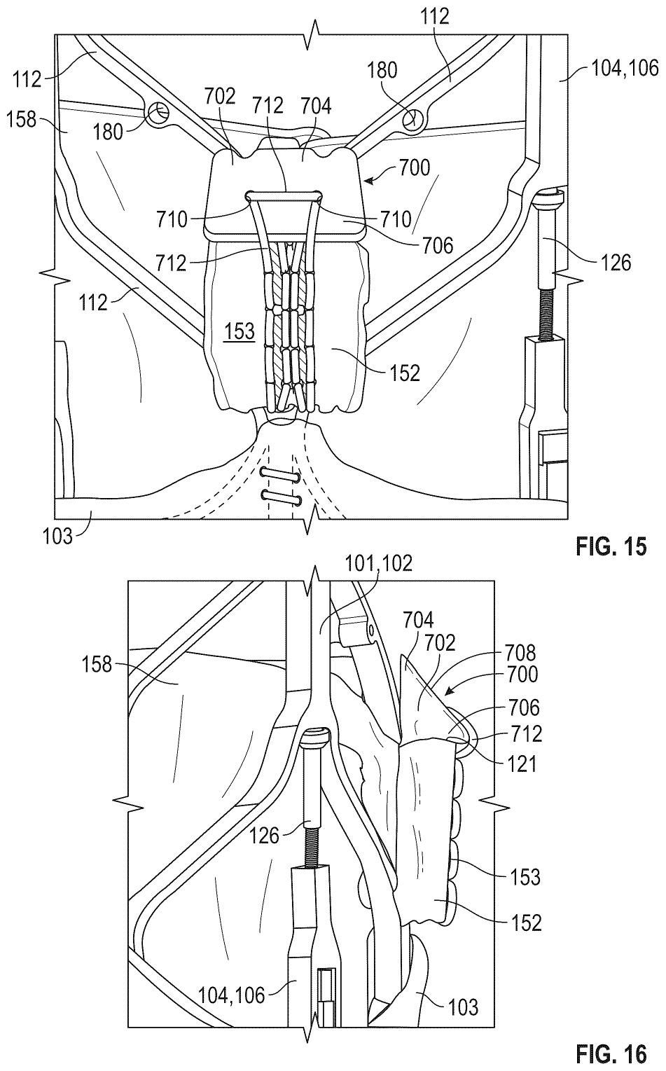

FIGS. 15-16 illustrate another example of a commissure cover 700, useable in addition to or in lieu of the commissure covers described previously. Commissure cover 700 can be configured as a wedge or ramp member having a wedge-shaped main body 702. Commissure cover 700 can function similarly to the commissure covers described previously. Namely, commissure cover 700 provides a ramped surface 708 that covers the outflow edge 121 of the commissure 152 such that the distal edge of a sheath (for example, sheath 216) advanced over the prosthetic valve 100 does not engage the outflow edge 121 of the commissure.

As best seen in FIG. 16, the main body 702 can have a first end portion 704 (or apical portion) having a first thickness, and a second end portion 706 (or base portion) having a second thickness greater than the first thickness. The main body 702 of the ramp member can be positioned adjacent the outflow edge portion 121 of the commissure 152 such that the second end portion 706 abuts the outflow edge 121. The main body 702 can comprises a ramped surface 708 extending from the first end portion 704 to the second end portion 706. When the main body 702 is secured to the frame 102, the ramped surface 708 extends from the radial outer surface 101 of the frame 102 to the radial outer surface 153 of the commissure 152, as shown in FIG. 16.

As shown in FIGS. 15-16, in the illustrated example, the main body 702 can have a triangular prism shape, for example, a right triangular prism. In some examples, the main body 702 can have any of various other shapes provided a ramped surface is defined that extends from the radial outer surface 101 of the frame 102 to the radial outer surface 153 of the commissure 152. In the illustrated example, the width of the first end portion 704 is less than the width of the second end portion 706 such that the ramp tapers from the second end portion to the first end portion. In some examples, the first width can be greater than the second width.

In some examples, the main body 702 can comprise a rigid material, such as any rigid implantable polymer or metal, for example, PET, PTFE, Cobalt Chrome (CoCr), etc.

Referring to FIG. 15, in some examples, the ramp member 700 can comprise one or more apertures 710 extending through the thickness of the main body 702. The apertures 710 can be used to secure the main body 702 to the commissure 152 and/or the frame 102, for example, using sutures 712 that extend through the apertures 710. In the illustrated example, the main body 702 comprises two apertures 710. However, in some examples, the main body 702 can comprise any number of apertures 710 as needed to secure the main body 702 to the commissure 152 and/or the frame 102 at the selected position.

During insertion of the radially compressed prosthetic valve 100 into the sheath 216, the sheath 216 can advance along the ramped surface 708 such that the distal edge of the sheath 216 does not engage the outflow edge 121 of the commissure 152.

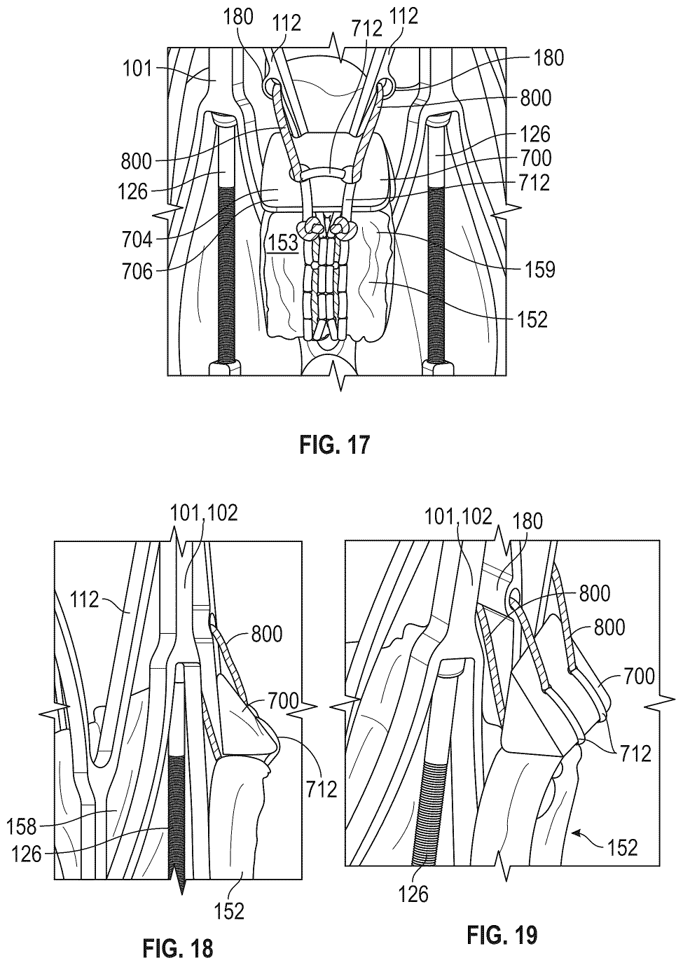

Referring to FIGS. 17-19, in some examples, the prosthetic heart valve 100 can further comprise one or more guide sutures 800. FIGS. 17-19 show the guide sutures with a patterned design for purposes of illustration. The pattern is added to distinguish the guide sutures 800 from the other sutures and does not represent actual surface ornamentation. The guide sutures 800 can be used in lieu of or in addition to any of the commissure covers described herein. For example, FIGS. 17-19 illustrate guide sutures 800 used with commissure cover 700 described previously and shown in FIGS. 15-16.

The guide sutures 800 can extend through a first end portion 159 of the commissure (for example, the outflow end) and can couple the first end portion 159 to the frame 102. In the illustrated example, the guide sutures 800 are coupled to eyelets 180 formed integrally as part of frame 102. In some examples, the guide sutures 800 can wrap around struts 112 of the frame 102 or be otherwise coupled to the frame. The illustrated example shows two guide sutures 800, however, in some examples, each commissure 152 can comprise a greater or fewer number of guide sutures.

In the example shown in FIGS. 17-19, the guide sutures 800 extend through the apertures 710 in the commissure cover 700. However, in some examples, the guide sutures 800 can extend from the commissure 152 over the commissure cover 700.

The guide sutures 800 can have a length configured such that when the frame 102 is in the radially compressed configuration, the guide sutures 800 are pulled taut. Such a configuration allows the guide sutures 800 to serve as a ramped surface (for example, in addition to the ramped surface 708 of the main body described above) extending from the radial outer surface 101 of the frame 102 to the radial outer surface 153 of the commissure 152. The guide sutures 800 can guide the distal edge of a shaft or sheath advancing over the prosthetic valve 100 onto the commissure cover 700, for example, such that the distal edge does not engage the apical end portion of the commissure cover 700.

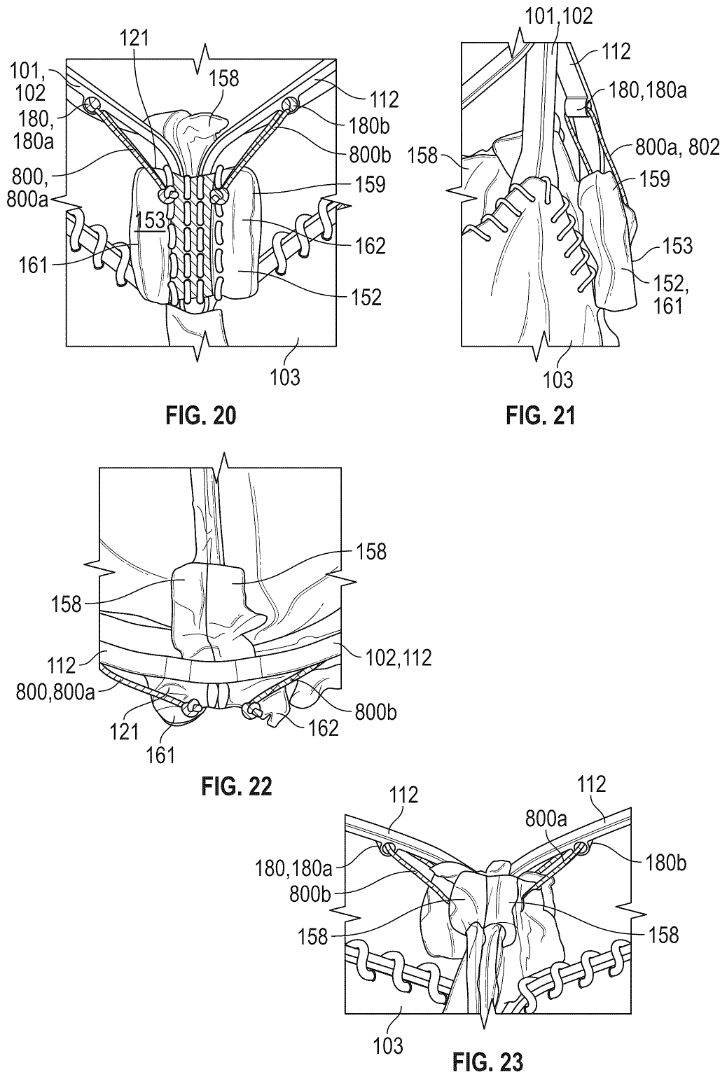

Referring to FIGS. 20-23, as mentioned previously, in some examples, the guide sutures 800 can be implemented in lieu of the commissure covers described herein. As shown in FIG. 20, in such examples, one or more guide sutures 800 can extend through a first end portion 159 of the commissure 152 (for example, the outflow end portion) and be coupled to the frame 102. The guide sutures 800 can define a ramped surface 802 (FIG. 21). That is, guide sutures 800 serve as a ramped surface 802 extending from the radial outer surface 101 of the frame 102 to the radial outer surface 153 of the commissure 152. The guide sutures 800 can guide the distal edge of a shaft or sheath advancing over the prosthetic valve 100 along the ramped surface 802, for example, such that the distal edge does not engage the outflow edge 121 of the commissure 152.

In the illustrated example, referring to FIG. 20, each commissure 152 can comprise first and second guide sutures 800a, 800b. The first guide suture 800a can extend through a first side portion 161 of the commissure 152 and be coupled to a first eyelet 180a of the frame 102 (for example, by extending through the eyelet). The second guide suture 800b can extend through a second side portion 162 of the commissure 152 and be coupled to a second eyelet 180b of the frame (for example, by extending through the eyelet). In some examples, a greater or fewer number of guide sutures can be used, for example, one, three, four, five, or six guide sutures. In examples with a greater number of guide sutures 800 more than one guide suture 800 can be coupled to each eyelet 180. For example, in an example with four guide sutures 800, first and second guide sutures can be sewn through the first side portion 161 of the commissure and third and fourth guide sutures can be sewn through the second side portion 162 of the commissure. The first and second guide sutures can be coupled to the first eyelet 180a and the third and fourth guide sutures can be coupled to the second eyelet 180b.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame; and
a commissure cover disposed at least partially over the outflow edge of the commissure, the commissure cover defining a ramped surface that extends from the radial outer surface of the frame to a radial outer surface of the commissure.

Example 2. The prosthetic heart valve of any example herein, particularly example 1, wherein each leaflet comprises two commissure tabs on opposite sides of the leaflet, and wherein each commissure is formed by pairing a commissure tab with an adjacent commissure tab of an adjacent leaflet.

Example 3. The prosthetic heart valve of any example herein, particularly any one of examples 1-2, wherein an outflow end portion of the commissure cover is coupled to one or more struts of the plurality of struts.

Example 4. The prosthetic heart valve of any example herein, particularly example 3, wherein the one or more struts of the plurality of struts are disposed at the outflow end of the frame.

Example 5. The prosthetic heart valve of any example herein, particularly any one of examples 1-4, wherein one or more struts of the plurality of struts comprise eyelets and wherein the commissure cover is coupled to the eyelets via one or more sutures extending through the eyelets.

Example 6. The prosthetic heart valve of any example herein, particularly any one of examples 1-5, wherein the commissure cover comprises a fabric member.

Example 7. The prosthetic heart valve of any example herein, particularly example 6, wherein the fabric comprises woven PET fabric.

Example 8. The prosthetic heart valve of any example herein, particularly any one of examples 1-7, further comprising an outer skirt disposed on a radial outer surface of the frame.

Example 9. The prosthetic heart valve of any example herein, particularly example 8, wherein an inflow end portion of the commissure cover is coupled to the outer skirt.

Example 10. The prosthetic heart valve of any example herein, particularly 8, wherein an inflow end portion of the commissure cover is formed integrally with the outer skirt.

Example 11. The prosthetic heart valve of any example herein, particularly any one of examples 1-10, wherein the ramped surface is pulled taut when the frame is in a radially compressed configuration.

Example 12. The prosthetic heart valve of any example herein, particularly any one of examples 1-11, wherein an outflow end portion of the commissure cover is rolled or folded to create a wedge member, and wherein the wedge member is disposed adjacent the outflow edge of the commissure to create the ramped surface.

Example 13. The prosthetic heart valve of any example herein, particularly any one of examples 1-12, wherein the commissure cover extends entirely over the radial outer surface of the commissure.

Example 14. The prosthetic heart valve of any example herein, particularly any one of examples 1-13, wherein the commissure cover has a rectangular shape.

Example 15. The prosthetic heart valve of any example herein, particularly any one of examples 1-14, wherein the commissure cover comprises a piece of material rolled or folded into a wedge shape.

Example 16. The prosthetic heart valve of any example herein, particularly example 15, wherein the commissure cover has a first end portion coupled to a first end portion of the commissure, and a second end portion coupled to the annular frame.

Example 17. The prosthetic heart valve of any example herein, particularly 16, wherein the first end portion of the commissure cover is an inflow end portion and the second end portion is an outflow end portion.

Example 18. The prosthetic heart valve of any example herein, particularly any one of examples 15-17, wherein the commissure cover is coupled to the prosthetic heart valve via a first suture extending through a first side portion of the commissure and diagonally over the commissure cover to couple a first strut of the frame, and a second suture extending through a second side portion of the commissure and diagonally over the commissure cover to couple a second strut of the frame.

Example 19. The prosthetic heart valve of any example herein, particularly any one of examples 15-17, wherein the commissure cover is coupled to the prosthetic valve via one or more sutures extending radially through the commissure cover and through a respective commissure opening.

Example 20. The prosthetic heart valve of any example herein, particularly example 19, wherein the sutures extending radially through the commissure opening are coupled to a wedge member disposed on a radial inner surface of the frame.

Example 21. The prosthetic heart valve of any example herein, particularly any one of examples 1-11, wherein the commissure cover comprises a V-shaped cutout at an outflow end portion, and wherein the outflow end portion is sutured to one or more struts of the frame along the V-shaped cutout.

Example 22. The prosthetic valve of any example herein, particularly example 21, wherein the V-shaped cutout is configured such that edges of the V-shape align with selected struts.

Example 23. The prosthetic heart valve of any one of claims 1-5, wherein the commissure cover comprises a ramp member.

Example 24. The prosthetic heart valve of any example herein, particularly example 23, wherein the ramp member comprises a rigid material.

Example 25. The prosthetic heart valve of any example herein, particularly any one of examples 23-24, wherein the ramp member comprises a first end portion having a first thickness and a second end portion having a second thickness greater than the first thickness.

Example 26. The prosthetic heart valve of any example herein, particularly example 25, wherein the second end portion of the ramp member is disposed directly adjacent the outflow edge of the commissure.

Example 27. The prosthetic heart valve of any example herein, particularly any one of examples 23-26, wherein the ramp member comprises one or more openings extending through a thickness of the ramp member.

Example 28. The prosthetic heart valve of any example herein, particularly example 27, wherein the ramp member is coupled to the commissure by one or more sutures extending through the one or more openings.

Example 29. The prosthetic heart valve of any example herein, particularly any one of examples 23-28, further comprising one or more guide sutures extending from the commissure to one or more eyelets of selected struts of the plurality of struts such that the guide sutures define a ramped surface from the eyelets to a radial outer surface of the commissure.

Example 30. The prosthetic heart valve of any example herein, particularly example 29, wherein the eyelets extend from the selected struts in a direction toward the inflow end of the frame.

Example 31. The prosthetic heart valve of any example herein, particularly any one of examples 29-30, wherein the selected struts are disposed at the outflow end of the frame.

Example 32. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame, wherein one or more selected struts of the plurality of struts comprise eyelets;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame;
a commissure cover disposed at least partially over the outflow edge of the commissure, the commissure cover having a first end portion and a second end portion and defining a ramped surface that extends from the radial outer surface of the frame to a radial outer surface of the commissure when the commissure cover is coupled to the frame;
an outer skirt disposed on the radial outer surface of the frame;
wherein the first end portion of the commissure cover is coupled to the eyelets by one or more sutures; and
wherein the second end portion of the commissure cover is coupled to the outer skirt by one or more sutures.

Example 33. The prosthetic heart valve of any example herein, particularly example 32, wherein each leaflet comprises two commissure tabs on opposite sides of the leaflet, and wherein each commissure is formed by pairing a commissure tab with an adjacent commissure tab of an adjacent leaflet.

Example 34. The prosthetic heart valve of any example herein, particularly any one of examples 32-33, wherein the eyelets extend from the selected struts toward the inflow end of the frame.

Example 35. The prosthetic heart valve of any example herein, particularly any one of examples 32-34, wherein the second end portion of the commissure cover comprises an inflow edge, a first side edge, and a second side edge, and wherein the inflow edge is coupled to the outer skirt.

Example 36. The prosthetic heart valve of any example herein, particularly any one of examples 32-35, wherein the one or more selected struts of the plurality of struts are disposed at the outflow end of the frame.

Example 37. The prosthetic heart valve of any example herein, particularly any one of examples 32-36, wherein the commissure cover comprises a fabric member.

Example 38. The prosthetic heart valve of any example herein, particularly example 37, wherein the fabric comprises woven PET fabric.

Example 39. The prosthetic heart valve of any example herein, particularly any one of examples 32-38, wherein the second end portion of the commissure cover is formed integrally with the outer skirt.

Example 40. The prosthetic heart valve of any example herein, particularly any one of examples 32-39, wherein the ramped surface is pulled taut when the frame is in a radially compressed configuration.

Example 41. The prosthetic heart valve of any example herein, particularly any one of examples 32-39, wherein the first end portion of the commissure cover is rolled or folded to define a wedge member, and wherein the wedge member is disposed adjacent the first edge of the commissure to create the ramped surface.

Example 42. The prosthetic heart valve of any example herein, particularly any one of examples 32-41, wherein the commissure cover extends entirely over the radial outer surface of the commissure.

Example 43. The prosthetic heart valve of any example herein, particularly any one of examples 32-42, wherein the commissure cover has a rectangular shape.

Example 44. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame; and
a commissure cover comprising a plurality of fabric layers arranged to define a ramp member, the commissure cover disposed adjacent the outflow edge of the commissure, wherein the radial outer surface of the commissure cover defines a ramped surface that extends from the radial outer surface of the frame to a radial outer surface of the commissure.

Example 45. The prosthetic heart valve of any example herein, particularly any one of examples 44, wherein the commissure cover has a first end portion coupled to a first end portion of the commissure, and a second end portion coupled to the annular frame.

Example 46. The prosthetic heart valve of any example herein, particularly example 45, wherein the first end portion of the commissure cover is an inflow end portion and the second end portion is an outflow end portion.

Example 47. The prosthetic heart valve of any example herein, particularly any one of examples 44-46, wherein the commissure cover is coupled to the prosthetic heart valve via a first suture extending through a first side portion of the commissure and diagonally over the commissure cover to couple a first strut of the frame, and a second suture extending through a second side portion of the commissure and diagonally over the commissure cover to couple a second strut of the frame.

Example 48. The prosthetic heart valve of any example herein, particularly example 47, wherein the first and second sutures are coupled to eyelets extending from selected struts of the plurality of struts.

Example 49. The prosthetic heart valve of any example herein, particularly any one of examples 45-46, wherein the commissure cover is coupled to the prosthetic valve via one or more sutures extending radially through the commissure cover and through a respective commissure opening.

Example 50. The prosthetic heart valve of any example herein, particularly example 49, wherein the sutures extending radially through the commissure opening are coupled to a wedge member disposed on a radial inner surface of the frame.

Example 51. The prosthetic heart valve of any example herein, particularly any one of examples 44-50, wherein the commissure cover extends at least partially over the outflow edge of the commissure.

Example 52. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame;
an outer skirt;
a commissure cover having a first end portion and a second end portion, the first end portion being coupled to the outer skirt via one or more sutures, the second end portion being folded to form a plurality of layers arranged to define a ramp member, the second end portion being positioned upstream adjacent the outflow edge of the commissure to define a ramped surface from the radial outer surface of the frame to the radial outer surface of the commissure.

Example 53. The prosthetic heart valve of any example herein, particularly example 52, wherein the commissure cover extends axially over the full length of the commissure.

Example 54. The prosthetic heart valve of any example herein, particularly any one of examples 52-53, wherein the second end portion of the commissure cover is coupled to the prosthetic heart valve via a first suture extending through a first side portion of the commissure and diagonally over the commissure cover to couple a first strut of the frame, and a second suture extending through a second side portion of the commissure and diagonally over the commissure cover to couple a second strut of the frame.

Example 55. The prosthetic heart valve of any example herein, particularly example 54, wherein the first and second sutures are coupled to eyelets extending from selected struts of the plurality of struts.

Example 56. The prosthetic heart valve of any example herein, particularly any one of examples 52-53, wherein the second end portion of the commissure cover is coupled to the prosthetic valve via one or more sutures extending radially through the second end portion of the commissure cover and through a respective commissure opening.

Example 57. The prosthetic heart valve of any example herein, particularly example 56, wherein the sutures extending radially through the second end portion of the commissure opening are coupled to a wedge member disposed on a radial inner surface of the frame.

Example 58. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame; and
a commissure cover comprising a rigid ramp member including one or more openings extending through a thickness of the ramp member, the ramp member disposed adjacent the outflow edge of the commissure and including a ramped surface that extends from the radial outer surface of the frame to the radial outer surface of the commissure.

Example 59. The prosthetic heart valve of any example herein, particularly any one of examples 58, wherein the ramp member comprises a first end portion having a first thickness and a second end portion having a second thickness greater than the first thickness.

Example 60. The prosthetic heart valve of any example herein, particularly example 59, wherein the second end portion of the ramp member is disposed directly adjacent the outflow edge of the commissure.

Example 61. The prosthetic heart valve of any example herein, particularly any one of examples 58-60, wherein the ramp member is coupled to the commissure by one or more sutures extending through the one or more openings.

Example 62. The prosthetic heart valve of any example herein, particularly any one of examples 58-61, wherein selected struts of the plurality of struts comprise eyelets extending from the selected struts in a direction toward the inflow end of the frame.

Example 63. The prosthetic heart valve of any example herein, particularly example 62, wherein the selected struts are disposed at the outflow end of the frame.

Example 64. The prosthetic heart valve of any example herein, particularly any one of examples 62-63, further comprising one or more guide sutures extending through the openings in the ramp member and through the eyelets such that the guide sutures define a ramped surface from the eyelets to the ramped surface of the ramp member.

Example 65. The prosthetic heart valve of any example herein, particularly any one of examples 58-64, wherein the ramp member comprises a triangular prism shape.

Example 66. The prosthetic heart valve of any example herein, particularly any one of examples 58-65, wherein the ramp member comprises a right triangular prism.

Example 67. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame, selected struts of the plurality of struts comprising eyelets extending from an inflow surface of the strut toward an inflow end portion of the frame;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame; and
one or more guide sutures extending through a respective commissure, the one or more guide sutures coupled to the eyelets of the frame such that the guide sutures define a ramped surface from the eyelets to a radial outer surface of the commissure.

Example 68. The prosthetic heart valve of any example herein, particularly example 67, wherein the one or more guide sutures comprise a first guide suture and a second guide suture.

Example 69. A method of loading a prosthetic heart valve into a shaft, comprising:
radially compressing a prosthetic heart valve, the prosthetic heart valve comprising an annular frame having an inflow end, an outflow end, a plurality of struts, and a plurality of commissure support structures each defining a commissure opening positioned along a circumference of the annular frame, a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame, and a commissure cover disposed at least partially over the outflow edge of the commissure;
as the frame is radially compressed, allowing the commissure cover to be pulled taut to define a ramped surface that extends from the radial outer surface of the frame to a radial outer surface of the commissure; and
advancing a shaft of a delivery apparatus over the radially compressed prosthetic valve such that a distal edge of the shaft advances along the ramped surface and does not engage the outflow edge of the commissure.

Example 70. A prosthetic heart valve according to any example herein, wherein the prosthetic heart valve is sterilized.

Example 71. A method of sterilizing any of the prosthetic heart valves described herein.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic heart valve, comprising:
a radially expandable and compressible annular frame having an inflow end, an outflow end, a plurality of struts defining the annular frame, and a plurality of commissure support structures each defining a commissure opening positioned along a circumference of the annular frame;
a valvular structure mounted within the annular frame, the valvular structure comprising a plurality of leaflets and a plurality of commissures each extending radially through a respective commissure opening to attach the valvular structure to the frame, each commissure having an outflow edge that extends from a radial outer surface of the frame; and
a commissure cover disposed at least partially over the outflow edge of the commissure, the commissure cover defining a ramped surface that extends from the radial outer surface of the frame to a radial outer surface of the commissure.

2. The prosthetic heart valve of claim 1, wherein each leaflet comprises two commissure tabs on opposite sides of the leaflet, and wherein each commissure is formed by pairing a commissure tab with an adjacent commissure tab of an adjacent leaflet.

3. The prosthetic heart valve of claim 1, wherein an outflow end portion of the commissure cover is coupled to one or more struts of the plurality of struts.

4. The prosthetic heart valve of claim 3, wherein the one or more struts of the plurality of struts are disposed at the outflow end of the frame.

5. The prosthetic heart valve of claim 1, wherein one or more struts of the plurality of struts comprise eyelets and wherein the commissure cover is coupled to the eyelets via one or more sutures extending through the eyelets.

6. The prosthetic heart valve of claim 1, wherein the commissure cover comprises a fabric member.

7. The prosthetic heart valve of claim 1, further comprising an outer skirt disposed on a radial outer surface of the frame.

8. The prosthetic heart valve of claim 7, wherein an inflow end portion of the commissure cover is coupled to the outer skirt.

9. The prosthetic heart valve of claim 7, wherein an inflow end portion of the commissure cover is formed integrally with the outer skirt.

10. The prosthetic heart valve of claim 1, wherein the ramped surface is pulled taut when the frame is in a radially compressed configuration.

11. The prosthetic heart valve of claim 1, wherein an outflow end portion of the commissure cover is rolled or folded to create a wedge member, and wherein the wedge member is disposed adjacent the outflow edge of the commissure to create the ramped surface.

12. The prosthetic heart valve of claim 1, wherein the commissure cover extends entirely over the radial outer surface of the commissure.

13. The prosthetic heart valve of claim 1, wherein the commissure cover comprises a piece of material rolled or folded into a wedge shape.

14. The prosthetic heart valve of claim 1, wherein the commissure cover has a first end portion coupled to a first end portion of the commissure, and a second end portion coupled to the annular frame.

15. The prosthetic heart valve of claim 14, wherein the commissure cover is coupled to the prosthetic heart valve via a first suture extending through a first side portion of the commissure and diagonally over the commissure cover to couple a first strut of the frame, and a second suture extending through a second side portion of the commissure and diagonally over the commissure cover to couple a second strut of the frame.

16. The prosthetic heart valve of claim 1, wherein the commissure cover comprises a V-shaped cutout at an outflow end portion, and wherein the outflow end portion is sutured to one or more struts of the frame along the V-shaped cutout.

17. The prosthetic valve of claim 16, wherein the V-shaped cutout is configured such that edges of the V-shape align with selected struts.

18. The prosthetic heart valve of claim 1, wherein the commissure cover comprises a ramp member.

19. The prosthetic heart valve of claim 18, wherein the ramp member comprises a first end portion having a first thickness and a second end portion having a second thickness greater than the first thickness.

20. The prosthetic heart valve of claim 19, wherein the second end portion of the ramp member is disposed directly adjacent the outflow edge of the commissure.

21. The prosthetic heart valve of claim 18, wherein the ramp member comprises one or more openings extending through a thickness of the ramp member.

22. The prosthetic heart valve of claim 21, wherein the ramp member is coupled to the commissure by one or more sutures extending through the one or more openings.

23. The prosthetic heart valve of claim 18, further comprising one or more guide sutures extending from the commissure to one or more eyelets of selected struts of the plurality of struts such that the guide sutures define a ramped surface from the eyelets to a radial outer surface of the commissure.

24. The prosthetic heart valve of claim 23, wherein the eyelets extend from the selected struts in a direction toward the inflow end of the frame.

25. The prosthetic heart valve of claim 23, wherein the selected struts are disposed at the outflow end of the frame.
